(19) United States
(12) Patent Application Publication
(10) Pub. No.: US 2016/0166844 A1
(43) Pub. Date: Jun. 16, 2016

(54) WEARABLE MAGNETIC DEVICE AND METHOD FOR SUBJECTING A BODY REGION TO A MAGNETIC FIELD

(71) Applicant: ESMO TECHNOLOGIES PTE. LTD., Singapore (SG)

(72) Inventor: Loh You CHUA, Singapore (SG)

(73) Assignee: ESMO TECHNOLOGIES PTE. LTD., Singapore (SG)

(21) Appl. No.: 14/902,645

(22) PCT Filed: Jul. 4, 2014
(86) PCT No.: PCT/SG2014/000322

§ 371 Date: Jan. 4, 2016

(30) Foreign Application Priority Data
Jul. 4, 2013 (SG) 201305196-6

(51) Int. Cl. A61N 2/06

(52) U.S. Cl. CPC A61N 2/06

(57) ABSTRACT

A wearable magnetic device, a method for subjecting a body region to a magnetic field and a set of parts. The wearable magnetic device comprises a magnetic structure configured to generate a magnetic field in a field area of the device; an auxiliary magnet connected to the magnetic structure and configured to increase the intensity of the magnetic field in the field area of the device; and a fastener; wherein the auxiliary magnet and the fastener are configured to magnetically secure the fastener on a side of the auxiliary magnet facing away from the field area of the device with a piece of fabric or a piece of garment disposed between the auxiliary magnet and the fastener.

### FIELD OF INVENTION

The present invention relates broadly to a wearable magnetic device, to a method for subjecting a body region to a magnetic field and to a set of parts.

### BACKGROUND

There is an ongoing demand for providing devices and methods for increased mental concentration and alertness and also for relief from ailments such as chronic aches and pain or injuries or post-surgery recovery or illnesses, and in a manner that is non-intrusive, that is comfortable, and that does not inhibit a person's activities.

While for example magnetic pendants and bracelets have been provided to try and address those needs, there are a number of problems associated with such devices. Those problems can include that the pendants or bracelets may not match a person's style of accessories, and/or that they are regarded as too obvious to others, which may cause embarrassment and/or a need to “have to explain” being felt by the wearer of such devices.

Embodiments of the present invention provide wearable devices, methods for subjecting a body region to a magnetic field and set of parts that seek to address at least one of the above problems.

### SUMMARY

In accordance with a first aspect of the present invention, there is provided a

[symbol]

In accordance with a first aspect, there is provided a wearable magnetic device comprising a magnetic structure configured to generate a magnetic field in a field area of the device; an auxiliary magnet connected to the magnetic structure and configured to increase the intensity of the magnetic field in the field area of the device; and a fastener; wherein the auxiliary magnet and the fastener are configured to magnetically secure the fastener on a side of the auxiliary magnet facing away from the field area of the device with a piece of fabric or a piece of garment disposed between the auxiliary magnet and the fastener.

In accordance with a second aspect, there is provided a method for subjecting a body region to a magnetic field, the method comprising providing a magnetic structure configured to generate a magnetic field in a field area; providing an auxiliary magnet connected to the magnetic structure and configured to increase the intensity of the magnetic field in the field area; and magnetically securing a fastener on a side of the auxiliary magnet facing away from the field area, with a piece of fabric or a piece of garment to be worn over the body region disposed between the auxiliary magnet and the fastener.

In accordance with a third aspect, there is provided a set of parts comprising a magnetic structure configured to generate a magnetic field in a field area of the device; an auxiliary magnet connected to the magnetic structure and configured to increase the intensity of the magnetic field in the field area of the device; one or more fasteners; and wherein the auxiliary magnet and the one or more fasteners are configured to magnetically secure the fastener on a side of the auxiliary magnet facing away from the field area of the device with a piece of fabric or a piece of garment disposed between the auxiliary magnet and the respective fastener.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1(a) is a schematic drawing illustrating a side view of a wearable magnetic device according to an example embodiment.

FIG. 1(b) is a schematic drawing illustrating a cross-sectional view of the wearable magnetic device of FIG. 1(a).

FIGS. 2(a) is a schematic drawing illustrating a portion of a magnetic structure of the device of FIG. 1(a).

FIGS. 2(b) is a schematic drawing illustrating a cross-sectional view of a portion of the device of FIG. 1(a).

FIGS. 3(a)-(c) are schematic drawings illustrating fabrication of a magnetic structure for a wearable magnetic device according to an example embodiment.

FIG. 4 is a schematic drawing illustrating a perspective side view of a magnetic structure for a wearable magnetic device according to an example embodiment.

FIG. 5 is a schematic drawing illustrating a back view of a shirt with one or more wearable magnetic devices according to example embodiments attached thereto.

FIG. 6 is a schematic drawing illustrating a back view of shorts with one or more wearable magnetic devices according to example embodiments attached thereto.

FIG. 7 shows a schematic drawing illustrating a cross-sectional side view of a portion of the shirt of FIG. 5 with one of the wearable magnetic devices attached.

FIG. 8 shows a schematic drawing illustrating a view of a waist belt or bandage with one of the wearable magnetic devices attached.

FIG. 9 shows a schematic drawing illustrating a view of a helmet or head dress with one of the wearable magnetic devices attached.

FIG. 10 shows comparative thermal images before and after 3 minutes exposure to a magnetic interference field of a neck region and a back region according to example embodiments.

FIG. 11 shows comparative thermal images before and after 3 minutes exposure to a magnetic interference field of an arm region and a calf region according to example embodiments.

FIG. 12 shows comparative thermal images before and after 3 minutes exposure to a magnetic interference field of a wrist region and a stomach region according to example embodiments.

FIG. 13 shows comparative thermal images before and after 3 minutes exposure to a magnetic interference field of head region according to example embodiments.

FIG. 14 shows a flow-chart illustrating a method for subjecting a body region to a magnetic field according to one embodiment.

### DETAILED DESCRIPTION

Embodiments of the present invention relate to a wearable magnetic device, to a method for subjecting a body region to a magnetic field and to a set of parts. In one embodiment, a method for subjecting a body region to a magnetic field is provided, comprising providing a magnetic structure configured to generate a magnetic interference field in a field area, providing an auxiliary magnet connected to the magnetic structure and configured to increase the intensity of the magnetic interference field in the field area, and magnetically securing a fastener on a side of the auxiliary magnet facing away from the field area and with a piece of garment to be worn over the body region disposed between the auxiliary magnet and the fastener.

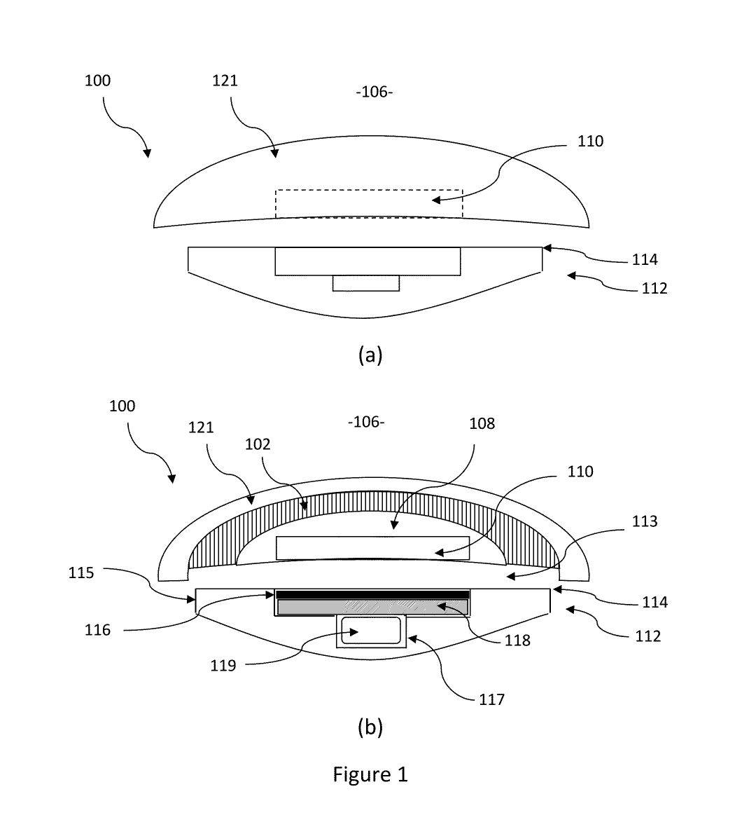

A schematic side view drawing and a schematic cross-sectional side view drawing of a magnetic device 100 according to an example embodiment are shown in FIGS. 1(a) and (b) respectively. The magnetic device 100 comprises a magnetic structure 102 configured to generate a magnetic interference field projecting in a field area 106 of the device 100. The device 100 further comprises a support structure 108 configured to support the magnetic structure 102, an auxiliary magnet 110 configured to increase the intensity of the magnetic interference field in the field area 106 of the device 100, and a fastener 112. The auxiliary magnet 110 and the fastener 112 are configured to magnetically secure the fastener 112 on a side of the auxiliary magnet 110 facing away from the field area of the device with a piece of fabric or a piece of garment (not shown) disposed between the auxiliary magnet 110 and the fastener 112. The multi-functional auxiliary magnet 110 in the example embodiment advantageously increases the magnetic interference field, as well as being configured for securing the device 100 to a fabric or a garment worn by a user. This can preferably provide for a convenient use of the device 100, and with little, if any, risk of damaging the fabric or garment. The magnetic structure 102, support structure 108 and auxiliary magnet 110 may be at least partially encapsulated using an adhesive sheet or sheets (not shown) such as, for example, cellophane tape or polyethylene tape, for structural integrity.

The auxiliary magnet 110 in the example embodiment is embedded in the support structure 108 and at a side thereof facing away from the field area 106 of the device 100. The fastener 112 is dimensioned such that an edge 114 of the fastener 112 extends beyond a circumference of the auxiliary magnet 110 in the magnetically fastened state but such that it is smaller than an internal recess area 113 of the device 100. This can advantageously facilitate a firm interlocking of the fastener 112 within the recess area 113 under magnetic attraction to the auxiliary magnet 110 with the piece of fabric or the piece of garment disposed there between.

The fastener 112 comprises a first rigid plastic disk 115, with a stepped recess area 117 formed therein. The fastener 112 further includes a flat plastic disk 116 disposed over a first, larger magnet 118 sitting on top of a second, smaller magnet 119. The rigid plastic disk 116 is fitted into the top part of the stepped recess area 117 such that it holds both magnets 118, 119 securely in place within the plastic disk 115.

The fastener 112 is preferably provided in a neutral color on at least the surface facing away from the field area, i.e. the surface that may be exposed to view when the magnetic device is worn by a user. In different embodiments, one or more fasteners 112 in different colors can be provided, for example as a set, enabling color co-ordination with a desired garment. This can advantageously facilitate a blending in with a garment worn by the user of the device 100, which can reduce chances of the wearing of the magnetic device being noted by others.

In this embodiment, the device 100 further comprises a rigid structure 121 made from plastic. The rigid plastic 121 substantially follows a dome-shaped contour of the surface of the magnetic structure 102 facing the field area 106 of the device 100 in the example embodiment. Because the device 100 comprises the plastic structure 121, a direct or close contact between the skin of the user and the magnetic structure can preferably be avoided. Through selection of the properties of the cover material, the device can be optimized for wearer comfort in terms of for example contact pressure, scratching, and/or skin irritation.

The rigid structure (or cover) 121 can have a diameter in a range from about 30-35 mm and a height in a range from about 8-12 mm. The disk 115 can have a diameter in a range from about 18-22 mm and a height in a range from about 5-8 mm. In one embodiment, the rigid structure (or cover) 121 has a diameter of about 33 mm and a height of about 10 mm, and the disk 115 has a diameter of about 20 mm and a height of about 6.5 mm. However, it will be appreciated that embodiments are not limited to those ranges and values, and that other dimensions and/or shapes may be used in different embodiments.

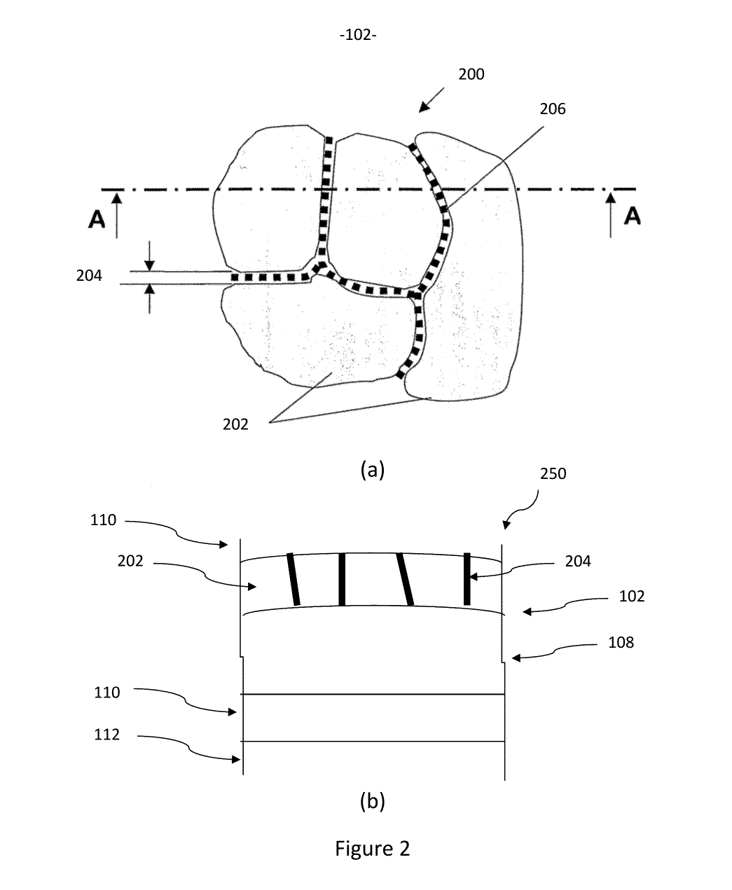

A schematic drawing of a portion 200 of the magnetic structure 102 of the device 100 is shown in FIG. 2(a). The relative dimensions of the various features of the magnetic structure 102 have been exaggerated for illustration purposes. The magnetic structure 102 generally comprises a plurality of magnet fragments 202 formed from a single piece of magnetic material (not shown) and movement of the magnet fragments 202 with respect to each other is inhibited. The plurality of magnet fragments 202 are spaced adjacent each other with a gap 204 defining a boundary 206 between adjoining magnet fragments 202 to produce a magnetic field created by magnetic interference of the magnet fragments 202. The size of the gap 204 between the magnet fragments 202 can for example range from about 0.05 mm to about 3.00 mm.

Magnetic interference of the magnet fragments 202 is found to enhance the strength of the magnetic field projected from the plurality of magnet fragments 202. Therefore, the greater the intensity of the magnetic interference, the greater the enhancement of the magnetic field strength.

A schematic drawing of a cross-section of a portion 250 of the device 100 along the plane of the sheet in FIG. 1(a) is shown in FIG. 2(b). The auxiliary magnet 110 is disposed on one side of the magnet fragments 202 of the magnetic structure 102. The auxiliary magnet 110 is connected to the magnet fragments via the support structure 108. The auxiliary magnet 110 helps to further increase an overall magnetic flux density of the magnetic field projected from the device 100 (FIG. 1(a)) and also helps to further increase the intensity of magnetic interference created at the gaps 204 between the magnet fragments 202. As the auxiliary magnet 110 is a single piece of magnetic structure in the example embodiment, there is no magnetic interference emitted from the auxiliary magnet 110 by itself. As a result, the auxiliary magnet 110 also preferably acts as a shield element to at least partially shield a magnetic interference field projecting from the side of the magnet fragments 202 facing the auxiliary magnet 110 from being emitted beyond the auxiliary magnet 110. The auxiliary magnet 110 can for example be in the form of a single piece of permanent magnet.

The magnetic material for forming the magnetic structure 102 and the auxiliary magnet 110 can be made of materials comprising, for example, ferrite, ceramics, samarium cobalt, or neodymium. The magnetic materials can either be polarized to the desired polarity before the magnetic structure 102 is formed or after the magnetic structure 102 is formed.

The intensity of magnetic interference created between the magnet fragments 202 depends on several factors and can be generally represented by the following equation:

Intensity of magnetic interference=f(B₁²,B₂²,L,g⁻²,D⁻²) (1)

where

B₁ is the average magnetic flux density of the magnet fragments 202 [Gauss];

B₂ is the magnetic flux density of the auxiliary magnet 110 [Gauss];

L is the total length of the boundary 204 between the magnet fragments [m];

g is the average gap distance between the magnet fragments [m], where g≠0; and

D is the perpendicular distance from a surface plane of the magnet fragments 202 [m], D≠0.

From the above equation (1), it is observed that at a given perpendicular distance (D) from a surface plane of the magnet fragments 202, the intensity of magnetic interference is proportional to the length of the boundary between the magnet fragments 202 (L) and the square of the average magnetic flux density (B₁) of the magnet fragments 202 and the square of the magnetic flux density (B₂) of the auxiliary magnet 110. However, the intensity of magnetic interference is inversely proportional to the square of the average gap distance (g) between the magnet fragments 202.

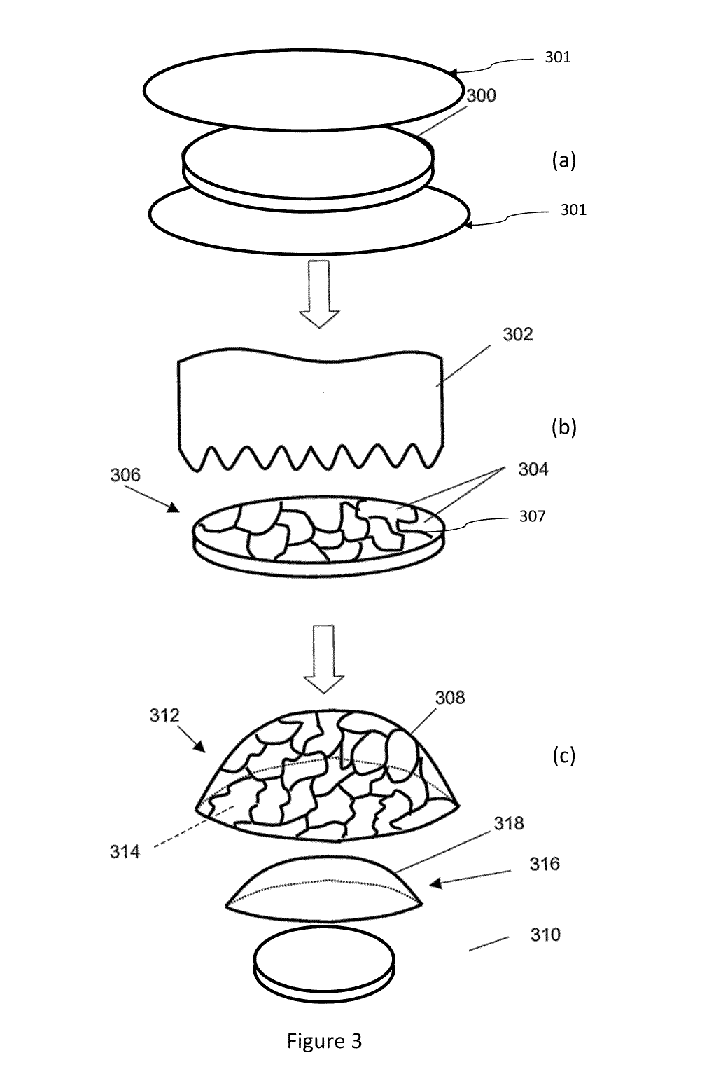

Schematic drawings illustrating a method of fabricating a magnetic structure for use in a device according to an example embodiment are shown in FIGS. 3(a) to 3(c). A fixture element in the form of two adhesive sheets 301 is attached onto a single disk of magnetic material 300. The piece of magnetic material 300 is disposed between the two adhesive sheets 301, as shown in FIG. 3(a). The adhesive sheets 301 are attached along opposing surfaces of the magnetic material 300.

The adhesive sheets 301 can comprise clear elastic adhesive sheets which are stretched and may be wound around the opposing surfaces of the magnetic material 300, thereby binding the magnetic material 300. As a result a compressive force is exerted on the magnetic material 300. However, it will be appreciated that other types of fixture elements and other methods of applying the fixture elements can be used, as long as movement of the magnet fragments 304 (FIG. 3(b)) with respect to each other is inhibited.

A punch 302 is used in this embodiment to physically break the piece of magnetic material 300 into a plurality of adjoining magnet fragments 304, as shown in FIG. 3(b). The punch 302 comprises a plurality of protrusions on a leading surface. The punch 302 is advanced towards the magnetic material 300 and applies a force onto the magnetic material 300 to break the magnetic material 300 into the plurality of adjoining magnet fragments 304. The punch 302 is retracted after the piece of magnetic material 300 is broken. Therefore, a magnetic structure 306 comprising the plurality of adjoining magnet fragments 304 is obtained. Movement of the magnet fragments 304 with respect to each other during the breaking of the magnetic material 300 is inhibited by the adhesive sheets 301 wound around the piece of magnetic material 300. The magnetic structure 306 of FIG. 3(b) is generally similar to the magnetic structure 200 illustrated in FIG. 2(a). The magnetic structure 306 is disk-shaped. It will be appreciated that the magnetic structure 308 can be of other shapes, e.g. square, circular, etc., depending on design requirements. Further, it will be appreciated that instead of having protrusions on the leading surface, the punch 302 can have other geometries and configurations, as long as the punch can break the magnetic material 300 into a plurality of magnet fragments 304. Also, other techniques for applying horizontal, lateral and/or bending stresses to fragment the magnetic material may be applied in different embodiments.

The adhesive sheets 301 serve to inhibit movement of the magnet fragments 304 with respect to each other by exerting a compressive force on the magnetic structure 306 to hold the magnet fragments 304 in place with respect to each other, against any repulsive forces between the magnet fragments 304. The magnet fragments 304 are spaced adjacent to each other with a separation gap defining a boundary 307 between adjoining magnet fragments 304 to produce a magnetic field created by magnetic interference. Further, the adhesive sheets 301 are preferably sufficiently deformable such that the adhesive sheets 301 are not or not substantially broken when force is applied to break the piece of magnetic material 300. The adhesive sheets 301 can be, for example, cellophane tape or polyethylene tape. In the above description, two adhesive sheets 301 are used, however, it will be appreciated that a single adhesive sheet can be attached along at least one surface of the piece of magnetic material 300 as long as the plurality of magnet fragments 304 can be held securely such that relative movement of the magnet fragments 304 is inhibited, thereby maintaining small gaps between the adjoining magnet fragments 304.

By keeping the plurality of magnet fragments 304 adjacent to each other with small gaps between adjoining magnet fragments 304, the magnetic interference created by the adjoining magnet fragments 304 is intensified. Referring to equation (1) (and assuming that all other factors, B₁, B₂, L and D are kept constant) it is observed that when the gap distance between adjoining magnet fragments 304 is decreased, the intensity of the magnetic interference is increased as the magnetic interference intensity is inversely proportional to the square of the gap distance (g). Therefore, the gap distance between adjoining magnet fragments 304 is preferably maintained as small as possible to achieve magnetic interference of a greater intensity.

The separation gaps between the magnet fragments 304 can, for example, be in the range of about 0.01 mm to about 3.00 mm. This advantageously creates a substantially intensified magnetic interference. The magnetic materials for forming the magnetic structure 306 can either be polarized to the desired polarity before the magnetic structure 306 is formed or after the magnetic structure 306 is formed.

Since increasing the intensity of magnetic interference increases the strength of the magnetic field, the size and/or the number of magnets required to achieve a desired magnetic field strength is reduced. This in turn can preferably reduce the total weight and cost of the device.

After breaking the piece of magnetic material 300, the generally planar magnetic structure 306 in FIG. 3(b) is formed into an dome-shaped magnetic structure 314, as shown in FIG. 3(c) while substantially maintaining a relative position of the magnet fragments 304 with respect to each other. The generally planar magnetic structure 306 is placed against a support 316 having a dome-shaped profile 318 such that the generally planar magnetic structure conforms to a dome-shaped profile 308. An adhesive sheet (not shown) can be used to wrap the magnetic structure 314 against the support 316 to maintain the shape of the dome-shaped magnetic structure 314. It will be appreciated that the generally planar magnetic structure 306 can be formed into other desired shapes such as an arc shape instead of a dome shape by using a support with a corresponding profile/shape. The support 316 can be made of any non-metallic material, such as plastic, in the example embodiment.

The resulting magnetic structure 312 comprises the dome-shaped magnetic structure 314 and a magnetic shielding device in the form of, for example, an auxiliary magnet 310 disposed on one side of the magnetic structure 314. In one embodiment, a south-pole side of the resulting magnetic structure 312 is made the convex side of the magnetic structure 314. The magnetic interference field projects from the convex south-pole side of the magnetic structure 314. The auxiliary magnet 310 is disposed at the other side (i.e. north-pole side) of the magnetic structure 314 to shield the magnetic interference field projecting from the north-pole side of the magnetic structure 314. In this example embodiment, a south-pole side of the auxiliary magnet 310 faces towards the north-pole side of the magnetic structure 314. The auxiliary magnet 310 may be any kind of permanent magnet or may be made of magnetic materials.

It will be appreciated that if a magnetic interference field from a north-pole is to be projected in the field area, the north-pole side of the magnetic structure can be the convex side and the south-pole side of the magnetic structure can be the other side. As will be appreciated by a person skilled in the art, there are two polarities and directions in a magnetic field. One direction is from the North magnetic pole and the other direction is from the South magnetic pole. Based on scientific convention, the compass “north” needle points in the direction of the magnetic flux, that is, in an outward direction from a magnet's North pole end and inward at the magnet's South pole end.

Two or more dome-shaped magnetic structures 314 may be stacked one on top of the other in other embodiments. Two or more auxiliary magnets 310 may be stacked one on top of the other in other embodiments.

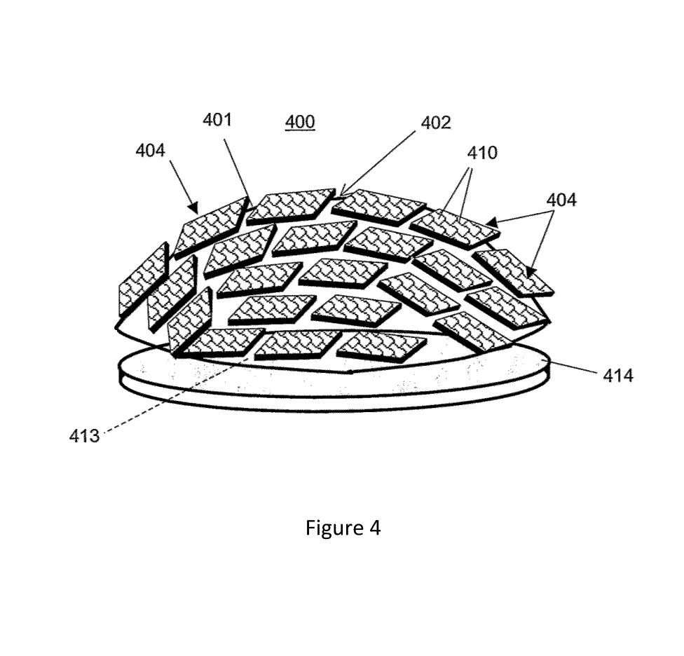

A schematic drawing of a magnetic device 400 according to another embodiment is shown in FIG. 4. The device 400 comprises a dome-shaped support 402, a plurality of substantially planar magnetic structures 404 arranged on a convex side 401 of the dome-shaped support 402. Each of the magnetic structures 404 in this embodiment is similar to, for example, the magnetic structure 308 of FIG. 3(b). Each magnetic structure 404 comprises a set of magnet fragments 310 formed from a single piece of magnetic material. An auxiliary magnet 414 is disposed on other side 413 of the dome-shaped support 402 to shield the magnetic interference field projecting from that side 413 of the support 402.

The magnetic structures 404 are arranged substantially in a staggered arrangement on the convex side 401 of the dome-shaped support 402 to produce further magnetic interference between the magnetic structures 404. This is in addition to the magnetic interference produced individually by the magnetic structures 404. It will be appreciated that in another embodiment, each planar magnetic structure 404 may be replaced by a stack of two or more planar magnetic structures.

Reference is also made to PCT Publication No. WO 2008/030191 for a description of fabrication methods suitable for producing magnetic structures configured to generate magnetic interference fields, the contents of which are hereby incorporated by cross-reference.

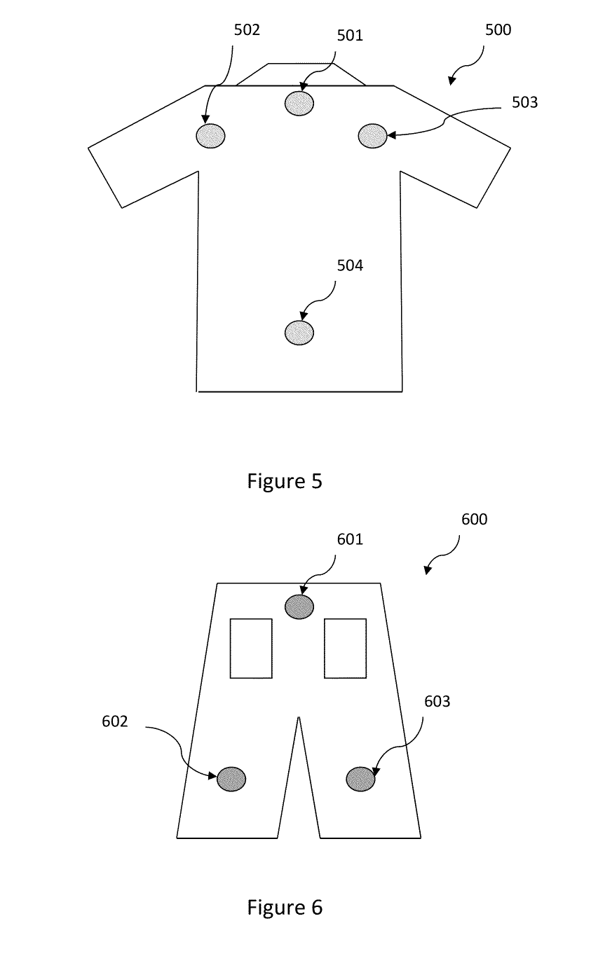

FIGS. 5 and 6 are schematic drawings illustrating back views of a shirt 500 and shorts 600 respectively, each with one or more magnetic devices according to example embodiments attached for subjecting different body regions of the wearer (not shown) to magnetic interference fields. By way of example, one or more of the neck region, shoulder regions and upper back region can be subjected to magnetic interference fields by one or more attached magnetic devices 501-504, and one or more of the lower back region and the thighs can be subjected to magnetic interference fields by one or more attached magnetic devices 601-603.

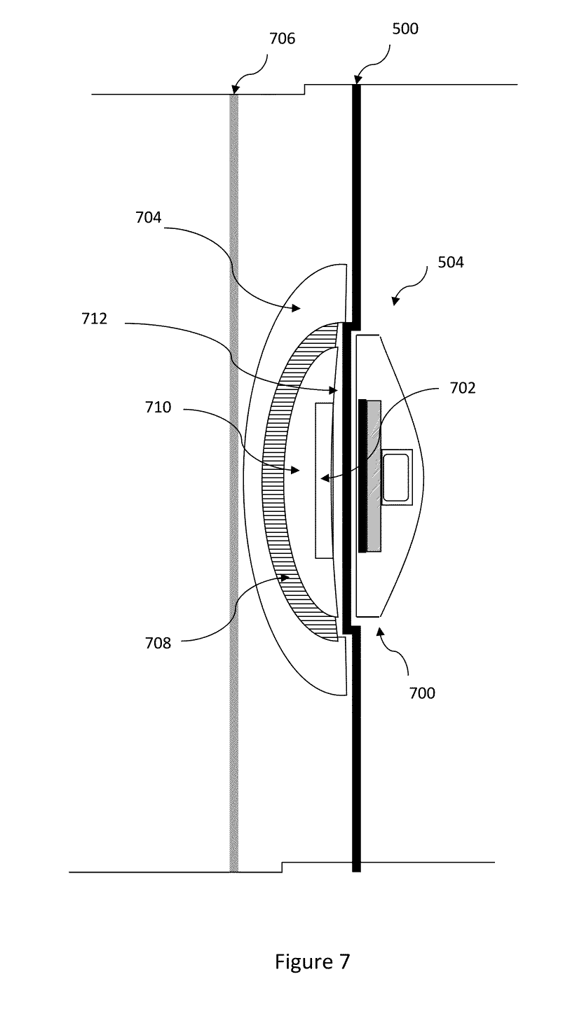

FIG. 7 shows a schematic drawing illustrating a cross-sectional side view of a portion of the shirt 500 with one of the wearable magnetic devices 504 attached. As can be seen from FIG. 7, a fastener 700 of the device 504 is magnetically secured on a side of an auxiliary magnet 702 of the facing away from the field area of the device 504, with a piece of the fabric of the shirt 500 disposed between the auxiliary magnet 702 and the fastener 700. A dome-shaped cover 704 of the device 504 is in contact with the skin 706 of the user or another garment worn underneath the shirt 500. A magnetic structure 708 and a support structure 710 of the device 504 are disposed underneath the cover 704. FIG. 7 also illustrates interlocking of the fastener 700 received in a recess 712 defined by the cover 704 with the piece of the fabric of the shirt 500 disposed between the auxiliary magnet 702 and the fastener 700 in the magnetically secured state. This can preferably facilitate avoiding undesired displacement of the device 504 during wearing.

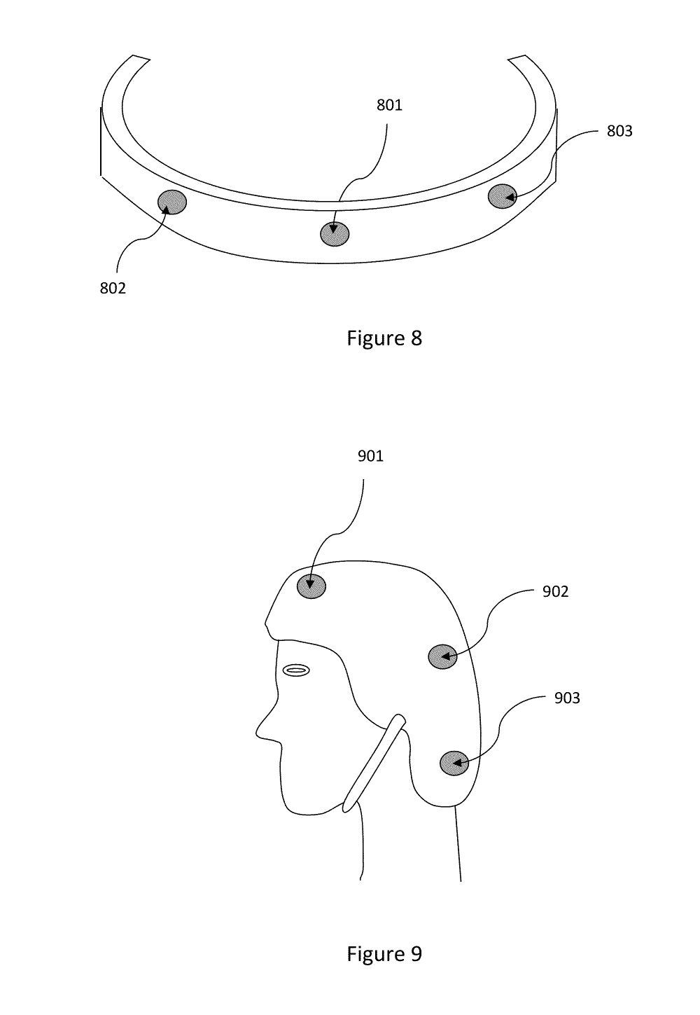

FIGS. 8 and 9 are schematic drawings illustrating s perspective view of a belt or bandage 800 and a side view of a helmet or head dress 900 respectively, each with one or more magnetic devices 801-803 and 901-903 respectively according to example embodiments attached for subjecting different body regions of a wearer to magnetic interference fields.

It will be appreciated that the magnetic device(s) according to embodiments of the present invention are not limited to the locations shown in FIGS. 5 to 9, but may instead be attached at different location(s) on the shirt 500, the shorts 600, the belt or bandage 800, the helmet or head dress 900 and/or any other garment worn or to be worn by a person for subjecting different body regions to magnetic interference fields. The phrase “piece of fabric or piece of garment” used in this specification and claims is intended to include, but is not limited to, the examples shown in FIGS. 5-9.

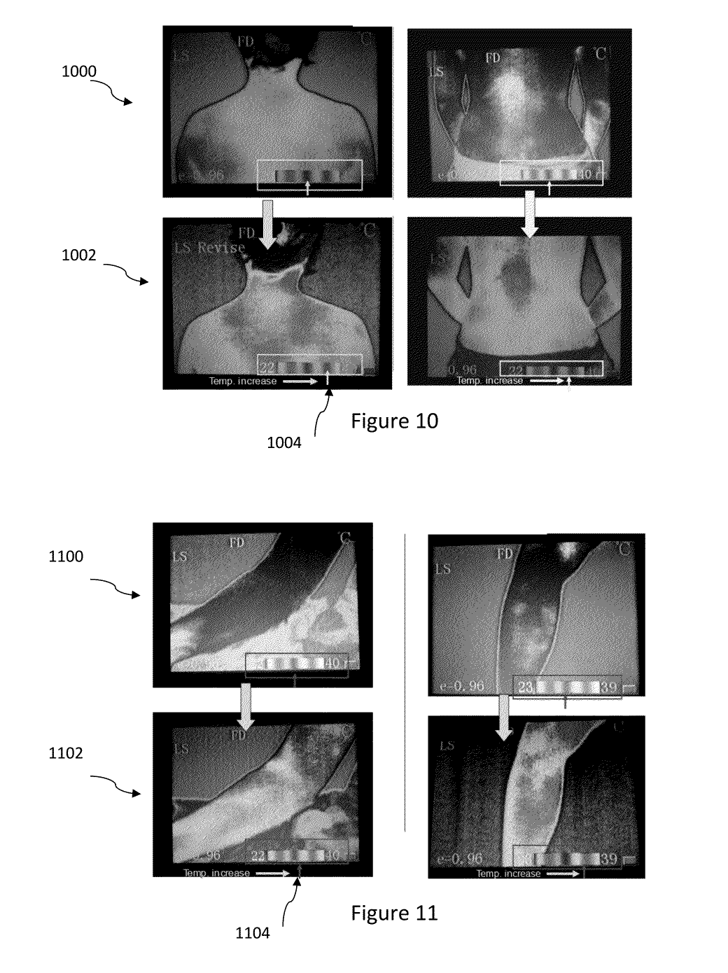

FIG. 10 shows comparative thermal images before (top row 1000) and after (bottom row 1002) 3 minutes exposure to a magnetic interference field of a neck region and a back region. The arrows e.g. 1004, 1006 indicate the corresponding surface temperature near the centers of the respective images.

FIG. 11 shows comparative thermal images before (top row 1100) and after (bottom row 1102) 3 minutes exposure to a magnetic interference field of an arm region and a calf region. The arrows e.g. 1104, 1106 indicate the corresponding surface temperature near the centers of the respective images.

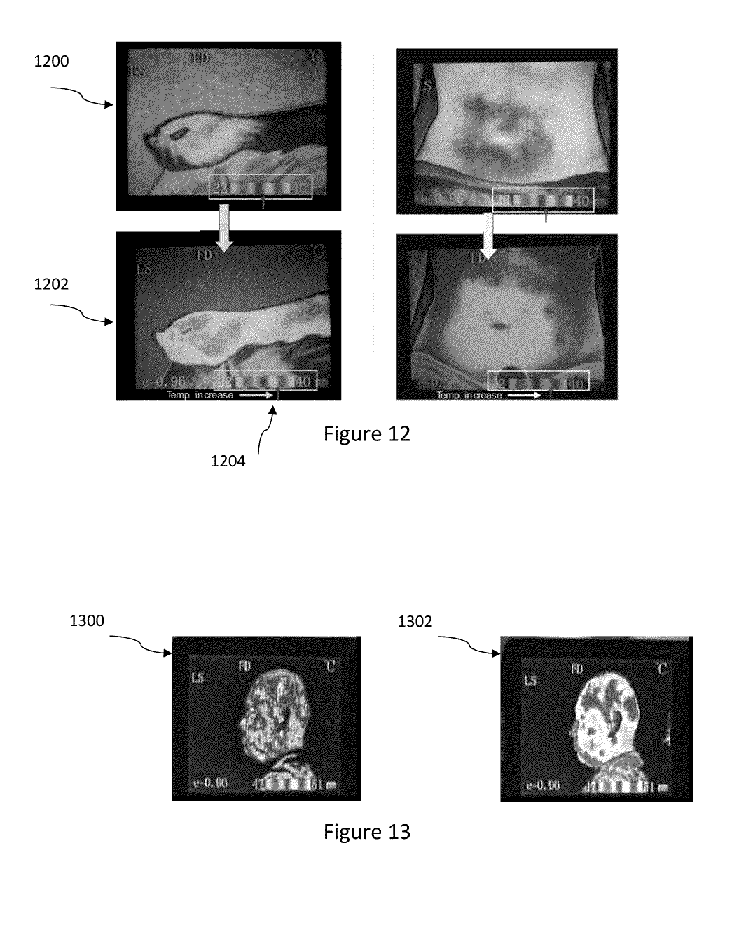

FIG. 12 shows comparative thermal images before (top row 1200) and after (bottom row 1202) 3 minutes exposure to a magnetic interference field of a wrist region and a stomach region. The arrows e.g. 1204, 1206 indicate the corresponding surface temperature near the centers of the respective images.

FIG. 13 shows comparative thermal images before (1300) and after (1302) 3 minutes exposure to a magnetic interference field of a head region. In the thermal images, the brighter shades indicate higher temperatures.

From FIGS. 10-13, it can be seen that the thermal infrared observations consistently show a marked increase in surface skin temperature by up to one degree Celsius when exposed to the static magnetic interference fields, of South-polarity in these experiments. This serves to demonstrate that there is a positive effect on improving the blood flow with static magnetic interference fields.

A possible explanation for the observed phenomenon is that the flux lines of the static magnetic interference fields may be in a chaotic and swirling form. This may create a magnetic induction effect on the liquids and water contained in the living tissues and blood vessels. The magnetic induction effect would consequently cause an increase in the water and liquids binding force, hence increasing the tissues and blood vessel wall tensioning.

Example embodiments of the present invention can advantageously be utilized for subjecting various body regions to magnetic interference fields, which can provide increased mental concentration and alertness and also for relief from ailments such as chronic aches and pain or injuries or post-surgery recovery or illnesses, and in a manner that is non-intrusive, that is comfortable, and that does not inhibit a person's activities.

In one embodiment, a wearable magnetic device comprises a magnetic structure configured to generate a magnetic field in a field area of the device; an auxiliary magnet connected to the magnetic structure and configured to increase the intensity of the magnetic field in the field area of the device; and a fastener; wherein the auxiliary magnet and the fastener are configured to magnetically secure the fastener on a side of the auxiliary magnet facing away from the field area of the device with a piece of fabric or a piece of garment disposed between the auxiliary magnet and the fastener.

The device can further comprise a support structure carrying the magnetic structure and disposed. The auxiliary magnet can be embedded in the support structure.

In one embodiment, the device further comprises a cover fitted to the magnetic structure and facing towards the field area of the device. The fastener is configured to be received in a recess defined by the cover with the piece of fabric or garment disposed between the auxiliary magnet and the fastener in a magnetically secured state.

In one embodiment, the fastener comprises a rigid body. The rigid body can comprise a recess formed therein for receiving at least one fastening magnet. The recess can comprise a stepped recess, and for receiving two fastening magnets of different size. The device can comprise the two magnets disposed in the stepped recess. The device can further comprising a rigid plate or disk fitted into a top portion of the stepped recess for securing the two magnets.

In one embodiment, the magnetic structure is configured to generate a magnetic interference field in the field area. The magnetic structure can comprise a plurality of fragments of a single piece magnetic material, wherein movement of the fragments with respect to each other is inhibited.

In another embodiment, the magnetic structure comprises two or more single piece magnetic materials stacked one on top of another, each single piece fragmented to form respective pluralities of fragments. The diameter and/or size and/or thickness of each piece of magnetic material and/or of the fragments in the respective pluralities of fragments can be the same or can be different.

In one embodiment, the auxiliary magnet is connected to the magnetic structure via a support for the magnetic structure.

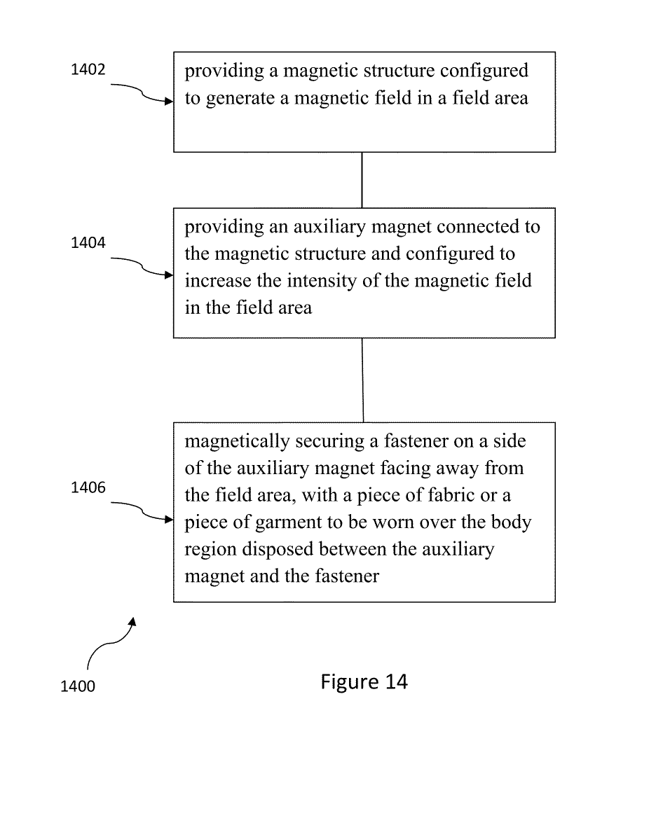

FIG. 14 shows a flow-chart 1400 illustrating a method for subjecting a body region to a magnetic field according to one embodiment. At 1402, a magnetic structure configured to generate a magnetic field in a field area is provided. At 1404, an auxiliary magnet connected to the magnetic structure and configured to increase the intensity of the magnetic field in the field area is provided. At 1406, a fastener is magnetically secured on a side of the auxiliary magnet facing away from the field area, with a piece of fabric or a piece of garment to be worn over the body region disposed between the auxiliary magnet and the fastener.

The magnetic field can comprise a magnetic interference field.

In one embodiment, the method can further comprise arresting a position of the magnetic structure relative to the piece of fabric or the piece of garment by receiving the fastener in a recess defined by a cover fitted to the magnetic structure with the piece of fabric or the piece of garment disposed between the auxiliary magnet and the fastener in a magnetically secured state.

In one embodiment, a set of parts comprises a magnetic structure configured to generate a magnetic field in a field area of the device; an auxiliary magnet connected to the magnetic structure and configured to increase the intensity of the magnetic field in the field area of the device; and one or more fasteners; wherein the auxiliary magnet and the one or more fasteners are configured to magnetically secure the fastener on a side of the auxiliary magnet facing away from the field area of the device with a piece of fabric or a piece of garment disposed between the auxiliary magnet and the respective fastener.

The set of parts can comprise two or more fasteners. The two or more fasteners can have different colors.

In one embodiment, the magnetic structure is configured to generate a magnetic interference field in the field area.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features, in particular any combination of features in the patent claims, even if the feature or combination of features is not explicitly specified in the patent claims or the present embodiments.

1. A wearable magnetic device comprising:
a magnetic structure configured to generate a magnetic field in a field area of the device;
an auxiliary magnet connected to the magnetic structure and configured to increase the intensity of the magnetic field in the field area of the device; and
a fastener;
wherein the auxiliary magnet and the fastener are configured to magnetically secure the fastener on a side of the auxiliary magnet facing away from the field area of the device with a piece of fabric or a piece of garment disposed between the auxiliary magnet and the fastener.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A method for subjecting a body region to a magnetic field, the method comprising:
providing a magnetic structure configured to generate a magnetic field in a field area;
providing an auxiliary magnet connected to the magnetic structure and configured to increase the intensity of the magnetic field in the field area; and
magnetically securing a fastener on a side of the auxiliary magnet facing away from the field area, with a piece of fabric or a piece of garment to be worn over the body region disposed between the auxiliary magnet and the fastener.

18. (canceled)

19. (canceled)

20. A set of parts comprising:
a magnetic structure configured to generate a magnetic field in a field area of the device;
an auxiliary magnet connected to the magnetic structure and configured to increase the intensity of the magnetic field in the field area of the device;
one or more fasteners; and
wherein the auxiliary magnet and the one or more fasteners are configured to magnetically secure the fastener on a side of the auxiliary magnet facing away from the field area of the device with a piece of fabric or a piece of garment disposed between the auxiliary magnet and the respective fastener.

21. (canceled)

22. (canceled)

23. (canceled)
